(19) United States
(12) Patent Application Publication
(10) Pub. No.: US 2010/0274160 A1
(43) Pub. Date: Oct. 28, 2010

(54) SWITCHING STRUCTURE AND SURGICAL EQUIPMENT

(76) Inventors: Chie Yachi, Fuchu-shi (JP); Yoshitaka Fujii, Atsugi-shi (JP)

Correspondence Address: OSTROLENK FABER GERB & SOFFEN, 1180 AVENUE OF THE AMERICAS, NEW YORK, NY, 100368403, US

(21) Appl. No.: 12/427,824

(22) Filed: Apr. 22, 2009

(51) Int. Cl. A61N 7/00; A61B 17/00; A61B 17/32

(52) U.S. Cl. 601/2; 606/1; 606/169

(57) ABSTRACT

A switching structure, which controls driving of equipment, includes a handle unit which is provided on the equipment and is held by a user, a switch operation section which is provided on the handle unit and includes a pressure reception portion which is pressed by the user, and a support portion which supports the pressure reception portion such that the pressure reception portion is pivotable about a pivotal center within the handle unit, relative to the handle unit, and also supports the pressure reception portion such that the pressure reception portion can be pushed from a desired direction toward the pivotal center of the pressure reception portion, when the user presses the pressure reception portion.

### BACKGROUND OF THE INVENTION

The present invention relates to a switching structure which controls driving of equipment, and surgical equipment having a switch thereof.

As a general example of medical equipment, there is disclosed an apparatus that is described in Jpn. Pat. Appln. KOKAI Publication No. 2002-291745 (patent document 1). A main body operation unit of this apparatus is provided with a switch which controls driving of the equipment. The switch is configured as a switch unit including a pressure reception portion which is pressed by a user, an operation rod of the pressure reception portion, and a switch contact. The pressure reception portion is provided at a distal end of the operation rod, and the switch contact is provided at a proximal end of the operation rod. At an operation time when the user performs an operation of pushing the pressure reception portion, the operation rod axially moves and presses the switch contact, thereby controlling the driving of the equipment.

### BRIEF SUMMARY OF THE INVENTION

According to an aspect of the present invention, there is provided a switching structure which controls driving of equipment, comprising: a handle unit which is provided on the equipment and is held by a user; a switch operation section which is provided on the handle unit and includes a pressure reception portion which is pressed by the user; and a support portion which supports the pressure reception portion such that the pressure reception portion is pivotable about a pivotal center within the handle unit, relative to the handle unit, and also supports the pressure reception portion such that the pressure reception portion can be pushed from a desired direction toward the pivotal center of the pressure reception portion, when the user presses the pressure reception portion.

Preferably, the switching structure further comprises a signal generation section which includes a switch contact and an urging member which holds the switch contact in an open state, and generates a signal for driving the equipment at a time when the switch contact is operated and closed, wherein the support portion holds, when the pressure reception portion is not pressed, the switch operation section at such a position that the switch contact is kept in the open state, and the support portion supports, when the pressure reception portion is pressed, the switch operation section such that the switch operation section can be moved against the urging member in such a direction as to close the switch contact.

Preferably, the switch operation section includes a shaft portion, a first coupling portion which is disposed on one end side of the shaft portion and is coupled to the pressure reception portion, and a second coupling portion which is disposed on the other end side of the shaft portion an is coupled to the handle unit on the pivotal center side, and the support portion includes a gap forming portion which forms, on the first coupling portion side, a gap for making the pressure reception portion movable in a pivotal direction about the pivotal center of the pressure reception portion.

Preferably, the gap forming portion includes an inclined surface with an opening gradually increasing on the pressure reception portion side, compared to the pivotal center side.

Preferably, the switch operation section pivots in a direction of pressing by the finger of the user who presses the pressure reception portion.

Preferably, the switch operation section is pivotable in a range of movement of the finger of the user who presses the pressure reception portion.

Preferably, the switch operation section is configured such that the pressure reception portion is pivotable in a state in which the shaft portion is inclined at least in two directions, i.e. a first direction and a second direction, from a neutral position of the shaft portion, the second coupling portion includes a first projection which projects in the first direction, a second projection which projects in the second direction, and a switch contact portion which is in contact with the signal generation section, the first projection abuts upon an inner surface of the handle unit in a case where the pressure reception portion is pivoted in the second direction, thus creating a first pivot point, and the switch contact portion presses the signal generation section in such a direction as to close the switch contact, in accordance with pivotal movement of the shaft portion about the first pivotal point, and the second projection abuts upon the inner surface of the handle unit in a case where the pressure reception portion is pivoted in the first direction, thus creating a second pivot point, and the switch contact portion presses the signal generation section in such a direction as to close the switch contact, in accordance with pivotal movement of the shaft portion about the second pivotal point.

Preferably, a press surface of the pressure reception portion has an arcuate shape along two directions, i.e. the first direction and the second direction.

Preferably, there are provided a plurality of the switch operation sections, and the handle unit is provided with a finger rest portion on which the user places the finger between the pressure reception portions of the plurality of switch operation sections.

Preferably, the switch operation section is configured such that the pressure reception portion is pivotable in a state in which the shaft portion is inclined in an arbitrary direction from a neutral position of the shaft portion, the second coupling portion includes a projection which projects in a circumferential direction of the shaft portion, and a switch contact portion which is in contact with the signal generation section, an end portion of the projection, which is located on a side opposite to a pivotal direction of the pressure reception portion, abuts upon an inner surface of the handle unit, thus creating a pivotal point, in a case where the pressure reception portion is pivoted in a direction other than a direction of a neutral position of the shaft portion, and the switch contact portion presses the signal generation section in such a direction as to close the switch contact, in accordance with pivotal movement of the shaft portion about the pivotal point.

According to another aspect of the invention, there is provided surgical equipment including a handpiece having a handle unit which is held by a surgeon, and a switch which is provided on the handle unit and controls driving of the equipment, wherein the switch includes: a switch operation section which includes a pressure reception portion which is pressed by a user; and a support portion which supports the pressure reception portion such that the pressure reception portion is pivotable about a pivotal center within the handle unit, relative to the handle unit, and also supports the pressure reception portion such that the pressure reception portion can be pushed from a desired direction toward the pivotal center of the pressure reception portion, when the user presses the pressure reception portion.

Preferably, the surgical equipment further comprises a signal generation section which includes a switch contact and an urging member which holds the switch contact in an open state, and generates a signal for driving the equipment at a time when the switch contact is operated and closed, wherein the support portion holds, when the pressure reception portion is not pressed, the switch operation section at such a position that the switch contact is kept in the open state, and the support portion supports, when the pressure reception portion is pressed, the switch operation section such that the switch operation section can be moved against the urging member in such a direction as to close the switch contact.

Preferably, the surgical equipment is an ultrasonic surgical apparatus comprising: an electrical connection section which electrically connects the handpiece and a generator which generates driving energy for driving the handpiece; an ultrasonic transducer which is provided in the handpiece and is electrically connected to the generator; and an ultrasonic probe which is connected to the ultrasonic transducer and to which ultrasonic vibration from the ultrasonic transducer is transmitted.

Preferably, the surgical equipment further comprises a sheath which covers the ultrasonic probe; and a jaw which is attached to a distal end portion of the sheath such that the jaw is pivotable relative to the distal end portion of the ultrasonic probe.

Preferably, the surgical equipment is a high-frequency surgical apparatus comprising: an electrical connection section which electrically connects the handpiece and a generator which generates driving energy for driving the handpiece; and an electrically conductive probe which is provided in the handpiece and is electrically connected to the generator, wherein a high-frequency current, which is supplied from the generator to the probe, is controllable by the switch.

Preferably, the surgical equipment is a surgical operation apparatus comprising: an electrical connection section which electrically connects the handpiece and a generator which generates driving energy for driving the handpiece; an ultrasonic transducer which is provided in the handpiece and is electrically connected to the generator; and an electrically conductive probe which is connected to the ultrasonic transducer and is electrically connected to the generator, wherein a high-frequency current, which is supplied from the generator to the probe, and a driving current for ultrasonic, which is supplied to the ultrasonic transducer, are controllable by the switch.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view which schematically shows the structure of the entirety of a handpiece of a surgical operation apparatus according to a first embodiment of the present invention;

FIG. 2 is a front view of the handpiece of the surgical operation apparatus according to the first embodiment;

FIG. 3 is a side view of a handle unit of the surgical operation apparatus according to the first embodiment;

FIG. 4 is a perspective view showing a disassembled state in which a coupling part of an assembly unit of the surgical operation apparatus according to the first embodiment is detached;

FIG. 5 is a longitudinal cross-sectional view taken in a lateral direction of the handpiece of the surgical operation apparatus according to the first embodiment;

FIG. 6 is a cross-sectional view taken along line VI-VI in FIG. 5;

FIG. 7 is a cross-sectional view taken along line VII-VII in FIG. 6;

FIG. 8 is a cross-sectional view taken along line VIII-VIII in FIG. 6;

FIG. 9 is a cross-sectional view taken along line IX-IX in FIG. 6;

FIG. 10 is a cross-sectional view taken along line X-X in FIG. 6;

FIG. 11 is a cross-sectional view taken along line XI-XI in FIG. 5;

FIG. 12 is a plan view showing a circuit pattern of a switch board of a hand switch in the handpiece of the surgical operation apparatus according to the first embodiment;

FIG. 13 is a longitudinal cross-sectional view showing a state in which a pressure reception portion of the hand switch in the handpiece of the surgical operation apparatus according to the first embodiment is held in a neutral position;

FIG. 14 is a longitudinal cross-sectional view showing a state in which the pressure reception portion of the hand switch in the handpiece of the surgical operation apparatus according to the first embodiment is pressed in an obliquely leftward direction;

FIG. 15 is a longitudinal cross-sectional view showing a state in which the pressure reception portion of the hand switch in the handpiece of the surgical operation apparatus according to the first embodiment is pressed in an obliquely rightward direction;

FIG. 16 is a perspective view showing a surgical operation apparatus according to a second embodiment of the present invention;

FIG. 17 is a longitudinal cross-sectional view showing a state in which a pressure reception portion of a hand switch in a handpiece of the surgical operation apparatus according to the second embodiment is held in a neutral position;

FIG. 18 is a perspective view showing the structure of a switch operation section of the hand switch in the handpiece of the surgical operation apparatus according to the second embodiment;

FIG. 19 is a side view showing a handpiece of a surgical operation apparatus according to a third embodiment of the present invention;

FIG. 20 is a side view showing a handpiece of a surgical operation apparatus according to a fourth embodiment of the present invention; and

FIG. 21 is a side view showing a handpiece of a surgical operation apparatus according to a fifth embodiment of the present invention.

### DETAILED DESCRIPTION OF THE INVENTION

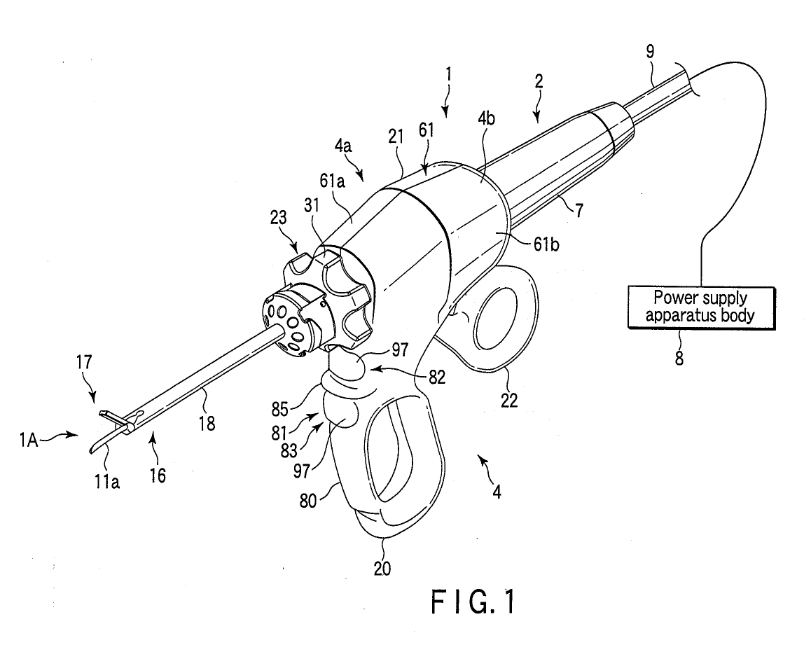

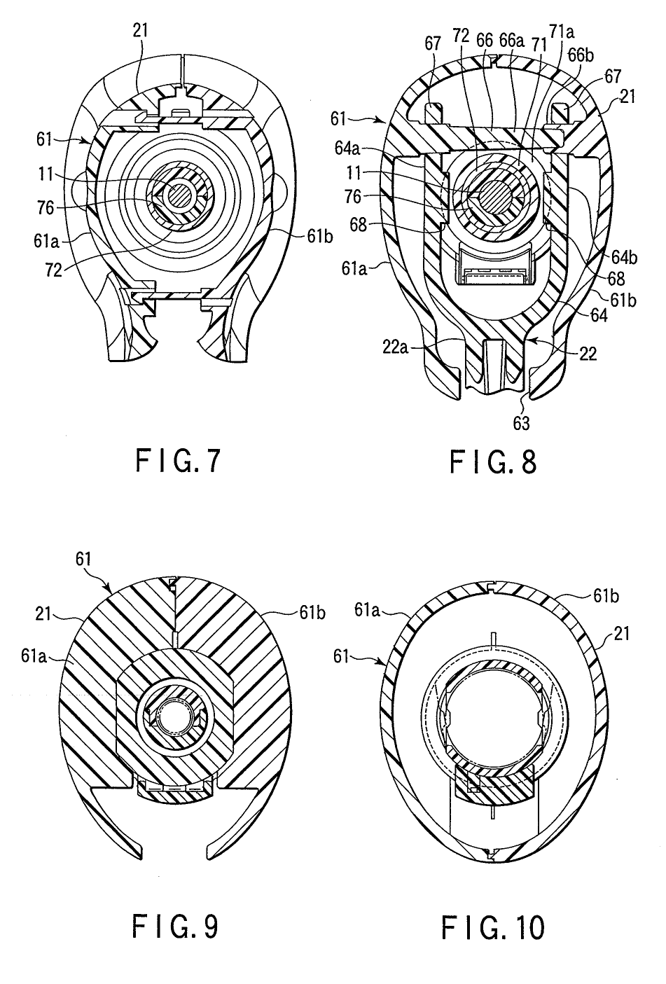

A first embodiment of the present invention will now be described with reference to FIG. 1 to FIG. 15. FIG. 1 schematically shows the structure of the entirety of a handpiece 1 of a surgical operation apparatus of the present embodiment. The surgical operation apparatus of this embodiment is an ultrasonic coagulation/incision therapeutic apparatus. This ultrasonic coagulation/incision therapeutic apparatus can perform therapeutic treatment such as incision, resection or coagulation, by using ultrasonic, and can also perform therapeutic treatment by high-frequency waves.

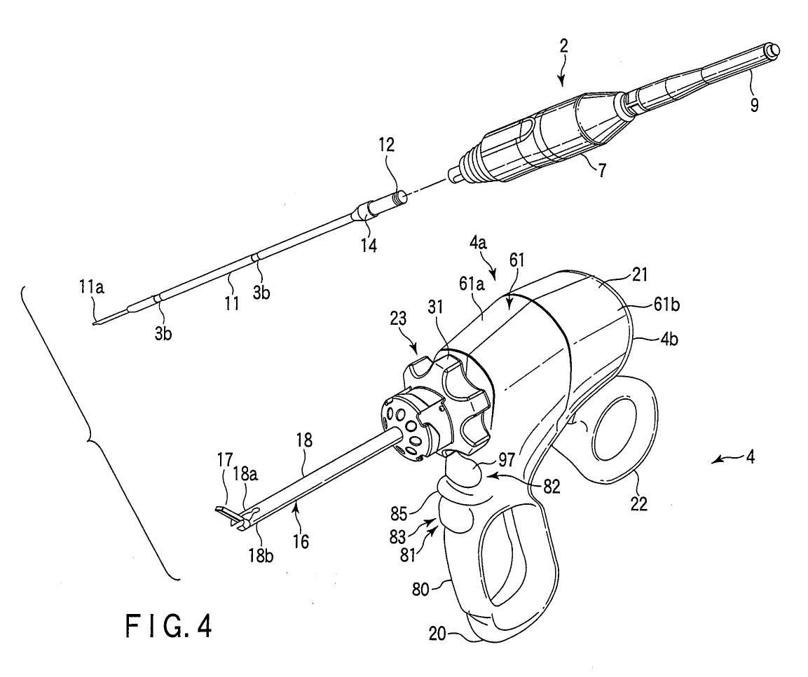

As is shown in FIG. 4, the handpiece 1 includes a transducer unit 2 and a handle unit (handle section) 4. These are detachably coupled.

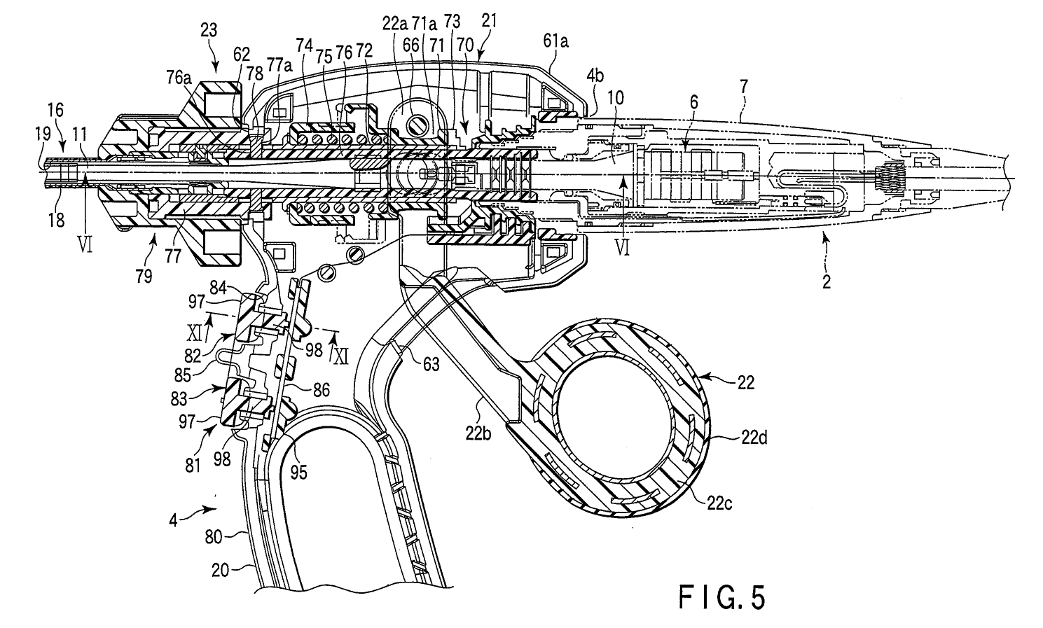

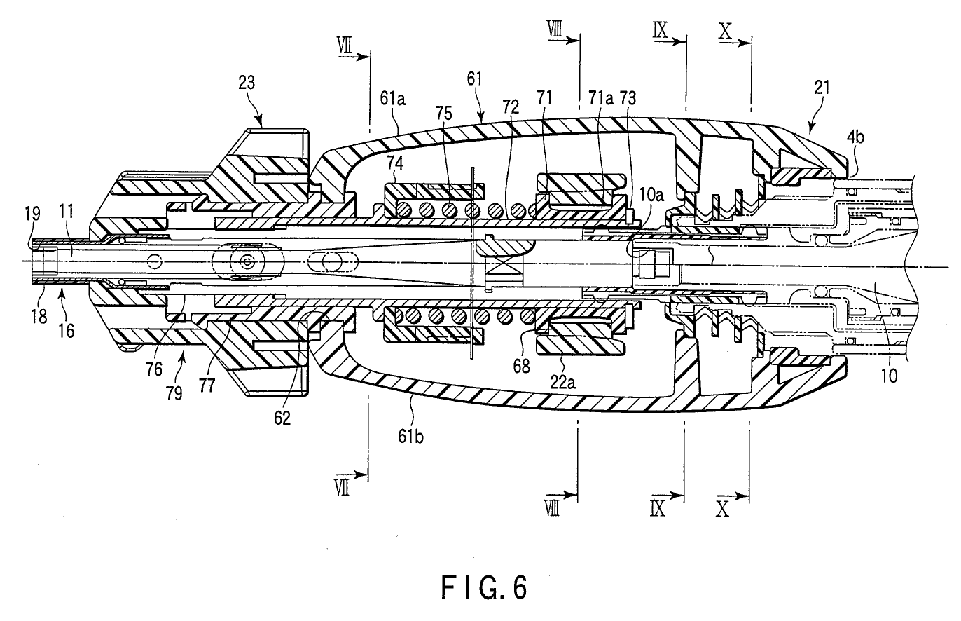

A transducer 6 (see FIG. 5) is assembled in the transducer unit 2. The transducer 6 generates ultrasonic vibration by a piezoelectric element which converts electric current to ultrasonic vibration. The outside of the transducer 6 is covered with a circular-cylindrical transducer cover 7. A cable 9 for supplying an electric current for generating ultrasonic vibration from a power supply apparatus body 8 extends from a rear end of the transducer unit 2. As shown in FIG. 5, a proximal end portion of a horn 10 for increasing the amplitude of ultrasonic vibration is coupled to a front end portion of the ultrasonic transducer 6 within the transducer cover 7. A screw hole portion 10a (see FIG. 6) for probe attachment is formed at a distal end portion of the horn 10.

An ultrasonic probe 11 is designed to have an entire length which is an integer-number of times of the half-wave length of ultrasonic vibration. A proximal end portion of the ultrasonic probe 11 is provided with a screw portion 12 for engagement with the screw hole portion 10a of the horn 10. The screw portion 12 of the ultrasonic probe 11 is engaged with the screw hole portion 10a of the horn 10 in the transducer unit 2. Thereby, the ultrasonic probe 11 and the transducer 6 are assembled. At this time, a high-frequency electric path (not shown), through which a high-frequency electric current flows, is formed in the coupled body between the ultrasonic transducer 6 and ultrasonic probe 11.

A distal end portion of the ultrasonic probe 11 is provided with a probe distal end 11a. The probe distal end 11a is formed in a substantially J-shaped curved shape. A flange portion 14 is provided at a node position of vibration which is located most on the proximal end side in the axial direction of the ultrasonic probe 11. Engagement recess portions (not shown) with a key groove shape are formed at three locations in the circumferential direction on the outer peripheral surface of the flange portion 14.

Further, the probe distal end 11a forms a first electrode portion, which is one of bipolar electrodes. The ultrasonic probe 11 has a cross-sectional area decreasing in the axial direction at several positions of nodes of vibration along the axial direction so as to obtain a necessary amplitude for therapeutic treatment at the probe distal end 11a. Rubber rings 3b, which are formed of elastic material in annular shapes, are attached to several positions of nodes of vibration along the axial direction of the ultrasonic probe 11. These rubber rings 3b prevent interference between the ultrasonic probe 11 and a sheath unit 5.

As shown in FIG. 4, the handle unit 4 comprises an elongated sheath body 16, a jaw 17 which is provided at a distal end portion of the sheath body 16, and an operation unit 4a which is provided at a proximal end portion of the sheath body 16. The operation unit 4a of the handle unit 4 is provided with a hold cylinder 21 having a substantially circular-cylindrical shape. A transducer connection portion 4b is formed at a proximal end portion of the hold cylinder 21.

The sheath body 16 includes a metallic outer sheath 18 which is an outer cylinder, and a metallic driving pipe (driving member) 19 which is an inner cylinder (inner sheath). The driving pipe 19 is axially movably inserted in the outer sheath 18. A proximal end portion of the outer sheath 18, together with a rotation operation knob 23 (to be described later), is attached to a distal end portion of the hold cylinder 21 so as to be rotatable about the center axis of the outer sheath 18.

A pair of left and right projection portions 77 are provided at a distal end portion of the outer sheath 18 so as to project to the front side of the outer sheath 18. Proximal end portions of the jaw 17 are rotatably attached to the respective projection portions 77 via pivot pins (not shown).

The jaw 17 is formed in a substantially J-shaped curved shape, which substantially corresponds to the curved shape of the probe distal end 11a in conformity to the curved shape of the probe distal end 11a of the ultrasonic probe 11. When the ultrasonic probe 11 and the handle unit 4 are assembled, the jaw 17 is disposed in a position to face the probe distal end 11a of the ultrasonic probe 11.

The jaw 17 includes a metallic jaw body (not shown) which is an electrically conductive member, and a hold member (not shown) which is attached to the jaw body. The hold member is composed of an electrode member for high-frequency therapeutic treatment, and a pad member for ultrasonic treatment. The electrode member forms a second electrode portion, which is the other of the bipolar electrodes. The pad member is formed of an insulator, for instance, a resin material such as polytetrafluoroethylene.

Further, a distal end portion of the driving pipe 19 is coupled to the jaw 17 via a coupling pin (not shown). The jaw body of the jaw 17 and the driving pipe 19 are formed to be electrically conductive via the coupling pin.

The outer peripheral surface of the outer sheath 18 is coated with an outer coating which is formed of an insulation material such as resin. An insulation tube, which is formed of an insulation material, is provided on the inner peripheral surface of the driving pipe 19. A proximal end portion of the insulation tube extends to the proximal end side of the sheath body 16. The driving pipe 19 and the ultrasonic probe 11 are electrically insulated by the insulation tube.

The operation unit 4a of the handle unit 4 is provided with a stationary handle 20 and a movable handle 22 which rotates. The handle unit 4 is provided with an operation unit housing 61 in which the hold cylinder 21 and the stationary handle 20 are formed integral by resin material. The operation unit housing 61, as shown in FIG. 6 to FIG. 10, includes two housing parts (left housing part 61a and right housing part 61b) which can be divided to the left and right from the center line position in the up-and-down direction. The left housing part 61a and right housing part 61b are detachably coupled.

On the front side of the hold cylinder 21, there is provided a knob hold portion 62 which holds the rotation operation knob 23. The rotation operation knob 23 is coupled to the knob hold portion 62 so as to be rotatable about the axis of the outer sheath 18.

A movable handle insertion hole 63, which is an elongated hole for insertion of the movable handle 22, is formed in an upper part of the rear surface of the stationary handle 20. As shown in FIG. 5, the movable handle 22 includes an upper arm 22a and a lower arm 22b. The upper arm 22a is disposed in a direction perpendicular to the axial direction of the ultrasonic probe 11. The lower arm 22b is bent obliquely downward to the rear side from a lower end portion of the upper arm 22a. A finger insertion ring portion 22c, in which the user's thumb, for example, is inserted, is formed at a terminal end portion of the lower arm 22b. A ring-shaped finger contact portion 22d of an elastic lining is provided on the finger insertion ring portion 22c.

The upper arm 22a is inserted into the operation unit housing 61 from the movable handle insertion hole 63 of the stationary handle 20. As shown in FIG. 8, the upper arm 22a includes, at an upper part thereof, a U-shaped coupling portion 64 having a substantially U shape. The U-shaped coupling portion 64 includes two arms 64a and 64b. The movable handle 22 is assembled to a slider member 71 in the state in which the slider member 71 of an operation force transmission mechanism 70 (to be described later) is inserted between the two arms 64a and 64b.

Pin insertion holes 67 of a pivot pin 66 are formed in upper end portions of the arms 64a and 64b, respectively. Further, operation pins 68 projecting inward are formed on the inner surface side below the pin insertion holes 67 of the arms 64a and 64b.

The pivot pin 66 is integrally formed on the inner surface side of the operation unit housing 61. A first shaft portion 66a, which constitutes the pivot pin 66, is formed on the left housing part 61a. A second shaft portion 66b serving as a bearing, in which a distal end of the first shaft portion 66a is inserted, is formed on the right housing part 61b. When the left housing part 61a and right housing part 61b are assembled, the distal end of the first shaft portion 66a is inserted and engaged in the second shaft portion 66b serving as the bearing.

The pivot pin 66 is inserted in the pin insertion holes 67 in the upper end portions of the arms 64a and 64b. Thereby, the upper end portion of the movable handle 22 is pivotally supported on the hold cylinder 21 via the pivot pin 66. The movable handle 22 rotates via the pivot pin 66, and the movable handle 22 is operated to be opened/closed relative to the stationary handle 20.

As shown in FIG. 5, an operation force transmission mechanism 70, which transmits the operation force of the movable handle 22 to the driving pipe 19 of the jaw 17, is provided within the hold cylinder 21. The operation force transmission mechanism 70 mainly comprises a metallic, circular-cylindrical slider receiving member 72, and the slider member 71. The slider receiving member 72 is disposed coaxial to the center axis of the hold cylinder 21, and extends in the same direction as the insertion direction of the ultrasonic probe 11.

A stopper 73 and a spring receiver 74 are provided on the outer peripheral surface of the slider receiving member 72. The stopper 73 is fixed to the outer peripheral surface of a proximal end portion of the slider receiving member 72. The spring receiver 74 is projectingly provided on the outer peripheral surface on a distal end side of the slider receiving member 72. The slider member 71 and a coil spring 75 are disposed between the stopper 73 and the spring receiver 74. The stopper 73 restricts the movement position of the rear end side of the slider member 71. A front end portion of the coil spring 75 abuts upon the spring receiver 74. The coil spring 75 is mounted between the spring receiver 74 and the slider member 71 with a predetermined amount of mounting force.

An annular engagement groove 71a is formed in a circumferential direction in the outer peripheral surface of the slider member 71. As shown in FIG. 6 and FIG. 8, the operation pins 68 of the movable handle 22 are inserted and engaged in the engagement groove 71a. If the movable handle 22 is grasped and the movable handle 22 is closed relative to the stationary handle 20, the operation pins 68 rotate about the pivot pin 66 in accordance with the rotational operation of the movable handle 22 at this time. In interlock with the operation of the operation pins 68, the slider member 71 moves forward in the axial direction of the ultrasonic probe 11. At this time, the slider receiving member 72, which is coupled to the slider member 71 via the coil spring 75, also moves forward and backward together with the slider member 71.

A tubular probe holder 76, which is formed of an insulation material and covers the ultrasonic probe 11, is provided within the slider receiving member 72. The probe holder 76 is fixed on the inner peripheral surface of the slider receiving member 72. The inner peripheral surface of the probe holder 76 and the outer peripheral surface of the ultrasonic probe 11 are fixed via a key groove (not shown) and a rotation prevention portion having a key. Thereby, the probe holder 76 is made movable in the axial direction relative to the ultrasonic probe 11, and the relative movement in the rotational direction of the probe holder 76 is restricted.

A proximal end portion of the driving pipe 19 is fixed to a distal end portion of the probe holder 76. Thereby, the operation force of the movable handle 22 is transmitted to the driving pipe 19 via the slider member 71, coil spring 75, slider receiving member 72 and probe holder 76, and the driving pipe 19 of the jaw 17 is axially moved. Thus, the jaw 17 is rotated via a pivot pin.

By the advancing/retreating operation of the driving pipe 19 in the axial direction, the driving force of the driving pipe 19 is transmitted to the jaw 17 via the coupling pin. Accordingly, the jaw 17 is rotated about the pivot pin. At this time, when the driving pipe 19 is pulled rearward, the jaw 17 is driven in a direction away from the probe distal end 11a about the pivot pin (“open position”). Conversely, when the driving pipe 19 is pushed forward, the jaw 17 is driven in a direction toward the probe distal end 11a about the pivot pin (“closed position”). By the rotation of the jaw 17 to the closed position, a living body tissue is held between the jaw 17 and the probe distal end 11a of the ultrasonic probe 11.

A therapeutic treatment section 1A of the handpiece 1 is formed by the jaw 17 and the probe distal end 11a of the ultrasonic probe 11. The therapeutic treatment section 1A is configured to select a plurality of therapeutic treatment functions, for example, two therapeutic treatment functions (a first therapeutic treatment function and a second therapeutic treatment function) in the present embodiment. For example, the first therapeutic treatment function is set so as to produce an ultrasonic therapeutic treatment output and a high-frequency therapeutic treatment output at the same time. The second therapeutic treatment function is set so as to produce only the high-frequency therapeutic treatment output.

The first therapeutic treatment function and the second therapeutic treatment function of the therapeutic treatment section 1A are not limited to the above-described configurations. For example, the first therapeutic treatment function may be set so as to produce an ultrasonic therapeutic treatment output at a maximum output state, and the second therapeutic treatment function may be set so as to produce the ultrasonic therapeutic treatment output at an arbitrarily set output state which is preset at a lower output state than the maximum output state.

A circular-cylindrical knob receiving member 77 is provided on the inner peripheral surface of the rotation operation knob 23. The inner peripheral surface of the rotation operation knob 23 and the outer peripheral surface of the knob receiving member 77 are fixed via a key groove (not shown) and a rotation prevention portion having a key, thereby to restrict relative movement in the rotational direction.

A proximal end portion of the knob receiving member 77 is coupled to the knob hold portion 62 at the front end portion of the hold cylinder 21 so as to be rotatable about the axis of the ultrasonic probe 11. Thereby, the rotation operation knob 23 is coupled to the knob hold portion 62 of the hold cylinder 21 so as to be rotatable about the axis of the ultrasonic probe 11.

A distal end portion of the probe holder 76 is provided in the knob receiving member 77. Pin insertion holes 77a and 76a, which extend in the radial direction, are formed in the knob receiving member 77 and the probe holder 76. A coupling pin 78 is inserted in the pin insertion hole 77a of the knob receiving member 77 and the insertion hole 76a of the probe holder 76. The knob receiving member 77 and probe holder 76 are fixed by the coupling pin 78, and relative movement in the rotational direction is restricted.

When the rotation operation knob 23 is operated and rotated, the knob receiving member 77 rotates together with the rotation operation knob 23. At this time, the rotation of the knob receiving member 77 is transmitted to the probe folder 76 via the coupling pin 78. Further, the rotation of the probe holder 76 is transmitted to the ultrasonic probe 11 via a key groove (not shown) and a rotation prevention portion having a key. Thus, the rotation of the rotation operation knob 23 is transmitted to the ultrasonic probe 11 via the knob receiving member 77, the coupling pin 78 and the probe holder 76 in succession, and the ultrasonic probe 11 rotates together with the rotation operation knob 23. The rotation operation knob 23, knob receiving member 77, coupling pin 78 and probe holder 76 constitute a rotation torque transmission section 79 which transmits rotation torque, which is produced by the rotation operation knob 23 in the direction about the axis of the ultrasonic probe 11, to the ultrasonic probe 11.

Furthermore, at the time of the rotational operation of the rotation operation knob 23, the outer sheath 18, together with the rotation operation knob 23, is rotated about the center axis of the outer sheath 18 relative to the hold cylinder 21. Thereby, at the time of the rotational operation of the rotation operation knob 23, the ultrasonic probe 11 and the outer sheath 18 of the sheath body 16 are simultaneously rotated in the direction about the center axis of the outer sheath 18. Accordingly, the probe distal end 11a of the ultrasonic probe 11 and the jaw 17 are rotated in the same direction at the same time.

The operation unit housing 61 includes a plural-finger insertion ring portion 80, in which the user's fingers other than the thumb, for example, are inserted, on the lower end side of the stationary handle 20. As shown in FIG. 5, the stationary handle 20 in the present embodiment includes a switch hold section 81 between the hold cylinder 21 and the plural-finger insertion ring portion 80.

As shown in FIG. 3, the switch hold section 81 includes, on the front side of the stationary handle 20, a switch attachment surface 84 to which a plurality of hand switches, for example, two hand switches (first switch 82 and second switch 83) in this embodiment, are attached. The first switch 82 and second switch 83 are switches for selecting the therapeutic treatment function of the therapeutic treatment section 1A of the handpiece 1.

In the switch hold section 81, the first switch 82 and second switch 83 are vertically arranged and disposed. Further, a finger rest portion 85, on which the user places the finger, is projectingly provided between the first switch 82 and second switch 83.

The first switch 82 is disposed on the upper side of the finger rest portion 85. A switch for selecting the first therapeutic treatment function, which is frequently used among the plural therapeutic treatment functions, is set as the first switch 82.

The second switch 83 is disposed on the lower side of the finger rest portion 85. A switch for selecting the second therapeutic treatment function, which is the other of the plural therapeutic treatment functions, is set as the second switch 83. For example, the first switch 82 is set as a switch button for coagulation/incision, and the second switch 83 is set as a switch button for coagulation.

The height of projection of the finger rest portion 85 from the switch attachment surface 84 is set to be greater than the height of projection of each of the first switch 82 and second switch 83 from the attachment surface 84. The finger rest portion 85 includes an extension portion 85a which continuously extends from the switch attachment surface 84 of the stationary handle 20 toward both sides.

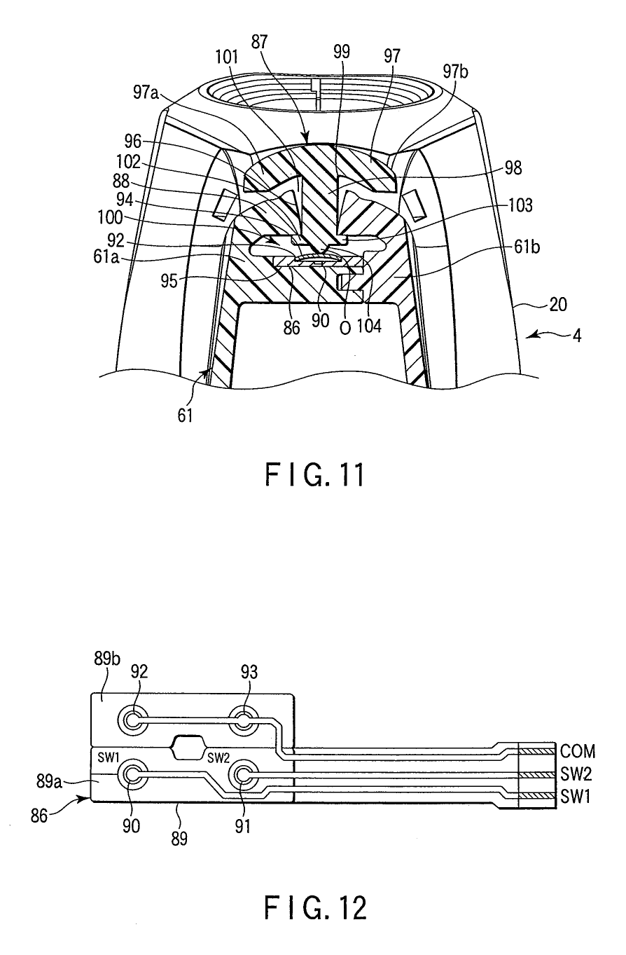

FIG. 11 schematically shows the structure of the first switch 82 of the handpiece 1. The second switch 83 has the same structure as the first switch 82. Thus, only the structure of the first switch 82 is described here, and a description of the structure of the second switch 83 is omitted.

The first switch 82 includes a switch board (signal generation section) 86, a switch operation section 87 and a support portion 88. The switch board 86 includes a flexible board sheet 89, as shown in FIG. 12. The board sheet 89 includes a sheet body 89a and a bent portion 89b. The sheet body 89a is provided with a first switch contact 90 for the first switch 82, and a second switch contact 91 for the second switch 83. The bent portion 89b is provided with a first common contact 92 for the first switch 82, and a second common contact 93 for the second switch 83. The board sheet 89 is used such that the bent portion 89b is bent over the sheet body 89a. Further, in the switch board 86, metal domes (urging members) 94, which are formed of a metallic material in a dome shape, are provided in association with the first switch 82 and second switch 83, respectively. The switch board 86 is configured such that the switch contact 90 is held in an open state by the metal dome 94. By the operation of the switch operation section 87, which is described later, the metal dome 94 is resiliently deformed in a collapsed state, so that the switch contact 90 is closed. Thereby, the first switch contact 90 of the first switch 82 and the first common contact 92 are connected, and a signal for driving equipment is generated from the first switch 82 of the switch board 86.

The operation unit housing 61 is provided with a board hold portion 95 which holds the switch board 86, and a switch operation section hold portion 96 which holds the switch operation section 87. The board hold portion 95 is disposed in the state in which the switch board 86 is held at the position corresponding to the first switch 82 and second switch 83 in the operation unit housing 61.

The switch operation section 87 includes a pressure reception portion 97 which is pressed by the user. The support portion 88 supports the pressure reception portion 97 such that the pressure reception portion 97 is pivotable about a pivotal center O within the operation unit housing 61, relative to the operation unit housing 61. Further, the support portion 88 supports the pressure reception portion 97 such that the pressure reception portion 97 can be pushed against the metal dome 94 in the direction of the pivotal center O of the pressure reception portion 97, when the user presses the pressure reception portion 97. When the pressure reception portion 97 is not pressed, the support portion 88 holds the switch operation section 87 at such a position that the switch contact 90 is kept in the open state.

The switch operation section 87 includes a shaft portion 98. A first coupling portion 99, which is coupled to the pressure reception portion 97, is disposed on one end side of the shaft portion 98. A second coupling portion 100, which is coupled to the operation unit housing 61 on the pivotal center O side, is disposed on the other end side of the shaft portion 98.

The support portion 88 includes a gap forming portion 101 which forms, on the first coupling portion 99 side of the switch operation section 87, a gap for making the pressure reception portion 97 movable in the pivotal direction about the pivotal center O of the pressure reception portion 97. The gap forming portion 101 includes an inclined surface 101a with an opening gradually increasing on the pressure reception portion 97 side, compared to the pivotal center O side.

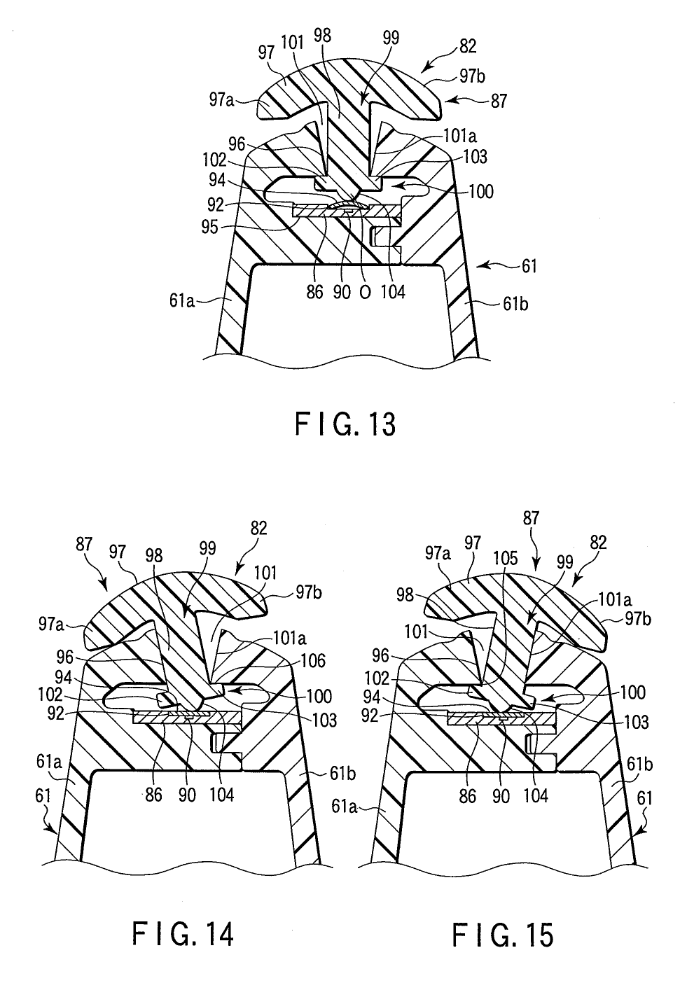

FIG. 13 shows the state in which the pressure reception portion 97 of the first switch 82 is held in a non-pressed state, and the shaft portion 98 of the pressure reception portion 97 is held in a neutral position. The shaft portion 98 of the pressure reception portion 97 is restricted in the state in which the shaft portion 98 is inclined only in two directions, namely, a first direction in which the shaft portion 98 is inclined from the neutral position to the left in FIG. 13, and a second direction in which the shaft portion 98 is inclined to the right in FIG. 13. As shown in FIG. 5, the wall surface of the operation unit housing 61 is disposed in sliding contact with the shaft portion 98 in a direction perpendicular to the pivotal direction of the shaft portion 98 of the pressure reception portion 97. Thereby, the pivotal direction of the pressure reception portion 97 is restricted to only the first direction in which the pressure reception portion 97 is inclined from the neutral position to the left in FIG. 13, and the second direction in which the pressure reception portion 97 is inclined to the right in FIG. 13.

The pressure reception portion 97 of the first switch 82 is an arcuate push button with a press surface having a shape which is elongated in a direction along the first direction and second direction. The first coupling portion 99 with the shaft portion 98 is disposed at a central position of the arcuate shape of the pressure reception portion 97. The pressure reception portion 97 includes a left extension portion 97a which extends from the first coupling portion 99 to the left in FIG. 13, and a right extension portion 97b which extends from the first coupling portion 99 to the right in FIG. 13.

The second coupling portion 100 includes a first projection 102 which projects in the first direction, a second projection 103 which projects in the second direction, and a switch contact portion 104 which is in contact with the metal dome 94 of the switch board 86.

In the case where the pressure reception portion 97 of the first switch 82 is operated and pushed straight at the neutral position in FIG. 13, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the switch contact 90 of the first switch 82 is closed.

In the case where the right extension portion 97b of the pressure reception portion 97 is pressed, the pressure reception portion 97 of the first switch 82 is pivoted in the second direction, as shown in FIG. 15. In this case, the first projection 102 abuts upon the inner surface of the operation unit housing 61, thus creating a first pivot point 105. At this time, in accordance with the pivotal movement of the shaft portion 98 about the first pivotal point 105, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the first switch contact 90 of the first switch 82 is closed.

In the case where the left extension portion 97a of the pressure reception portion 97 is pressed, the pressure reception portion 97 of the first switch 82 is pivoted in the first direction, as shown in FIG. 14. In this case, the second projection 103 abuts upon the inner surface of the operation unit housing 61, thus creating a second pivot point 106. At this time, in accordance with the pivotal movement of the shaft portion 98 about the second pivotal point 106, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the switch contact 90 of the first switch 82 is closed.

As regards the second switch 83, the same switching operation as with the first switch 82 is performed. Thereby, the metal dome 94 of the second switch 83 is resiliently deformed in a direction in which the metal dome 94 is collapsed. Thus, the second switch contact 91 of the second switch 82 and the second common contact 93 are connected, and the switch contact 91 of the second switch 83 is closed. Accordingly, a signal for driving equipment is generated from the second switch 83 of the switch board 86.

The operation unit housing 61 may be provided with a rubber cover which covers the entire outer peripheral surface of the pressure reception portion 97 of the first switch 82, and a rubber cover which covers the entire outer peripheral surface of the pressure reception portion 97 of the second switch 83. In this case, the first switch 82 and second switch 83 can be provided with waterproof seal structures.

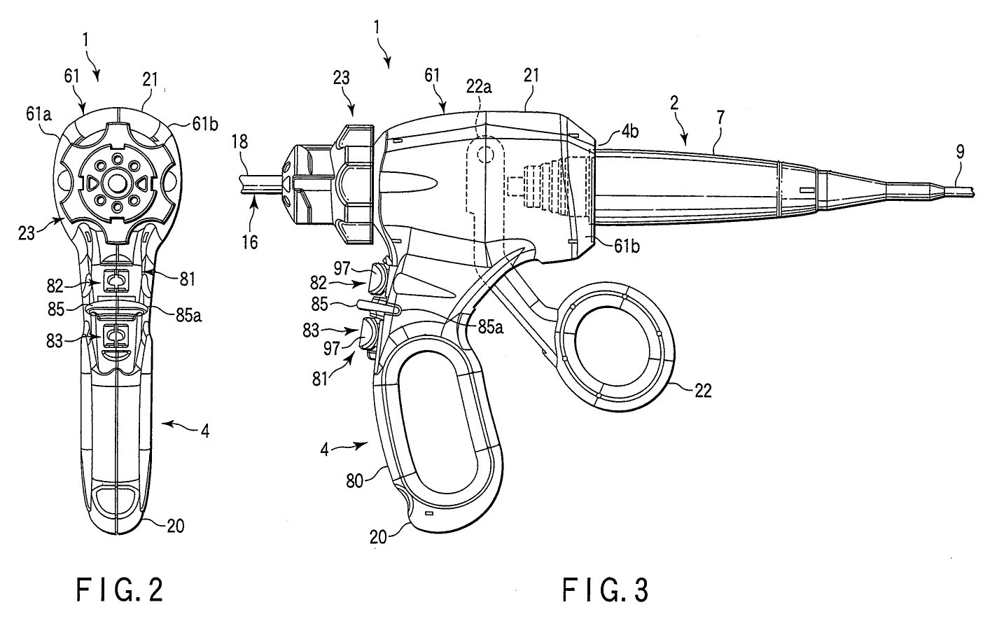

Next, the operation of the present embodiment is described. The handpiece 1 of the surgical operation apparatus according to the present embodiment is configured to be separable into two units, namely the transducer unit 2 and the handle unit 4, as shown in FIG. 2. When the handpiece 1 is used, the transducer unit 2 and handle unit 4 are coupled. Thereby, a first high-frequency electric path 13, through which high-frequency current is transmitted to the coupled body of the transducer unit 2 and ultrasonic probe 11, is formed.

On the other hand, a second high-frequency electric path, through which high-frequency current is transmitted to the electrode member of the jaw 17, is formed. Thereby, the second electrode portion of the bipolar electrodes is formed in the electrode member of the jaw 17, and the assembly work is finished.

When the handpiece 1 is used, the movable handle 22 is opened/closed relative to the stationary handle 20. In interlock with the operation of the movable handle 22, the driving pipe 19 is moved in the axial direction. In interlock with the advancing/retreating movement of the driving pipe 19 in the axial direction, the jaw 17 is opened/closed relative to the probe distal end 11a of the ultrasonic probe 11. In the case where the movable handle 22 is closed relative to the stationary handle 20, the driving pipe 19 is pushed forward in interlock with the operation of the movable handle 22. In interlock with the pushing operation of the driving pipe 19, the jaw 17 is driven in a direction toward the probe distal end 11a of the ultrasonic probe 11 (“closed position”). By the rotation of the jaw 17 to the closed position, a living body tissue is held between the jaw 17 and the probe distal end 11a of the ultrasonic probe 11.

In this state, one of the first switch 82 and second switch 83 of the stationary handle 20 is selectively operated and pressed. When the first switch 82 is pressed, a driving current is supplied to the ultrasonic transducer 6 at the same time as the supply of high-frequency power, and the ultrasonic transducer 6 is driven. At this time, ultrasonic vibration from the ultrasonic transducer 6 is transmitted to the probe distal end 11a via the vibration transmission member 11. Thereby, with the simultaneous use of the ultrasonic and the high-frequency power, therapeutic treatment such as incision or resection of the living body tissue is performed. In addition, a coagulation treatment of the living body tissue may also be performed with use of ultrasonic.

When the first switch 82 is pushed, if the pressure reception portion 97 of the first switch 82 is pushed straight at the neutral position in FIG. 13, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the switch contact 90 of the first switch 82 is closed. Accordingly, a signal for driving equipment is generated from the first switch 82 of the switch board 86.

In the case where the right extension portion 97b of the pressure reception portion 97 is pressed, the pressure reception portion 97 of the first switch 82 is pivoted in the second direction, as shown in FIG. 15. In this case, the first projection 102 abuts upon the inner surface of the operation unit housing 61, thus creating the first pivot point 105. At this time, in accordance with the pivotal movement of the shaft portion 98 about the first pivotal point 105, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the switch contact 90 of the first switch 82 is closed. Accordingly, a signal for driving equipment is generated from the first switch 82 of the switch board 86.

In the case where the left extension portion 97a of the pressure reception portion 97 is pressed, the pressure reception portion 97 of the first switch 82 is pivoted in the first direction, as shown in FIG. 14. In this case, the second projection 103 abuts upon the inner surface of the operation unit housing 61, thus creating the second pivot point 106. At this time, in accordance with the pivotal movement of the shaft portion 98 about the second pivotal point 106, the switch contact portion 104 presses the metal dome 94 of the switch board 86. Thereby, the metal dome 94 of the first switch 82 is resiliently deformed in a direction in which the metal dome 94 is collapsed. Thus, the first switch contact 90 of the first switch 82 and the first common contact 92 are connected, and the switch contact 90 of the first switch 82 is closed. Accordingly, a signal for driving equipment is generated from the first switch 82 of the switch board 86.

Therefore, in the present embodiment, no matter which position of the arcuate push button of the pressure reception portion 97 of the first switch 82 is pressed, it is possible to perform the associated switch operation, that is, the operation of generating the signal for driving equipment from the first switch 82 of the switch board 86.

In the case where the pressing operation of the pressure reception portion 97 of the first switch 82 is released, the metal dome 94 of the first switch 82 resiliently restores to its original dome shape, and the first switch point 90 of the first switch 82 and the first common contact 92 are disconnected. Accordingly, the switch contact 90 of the first switch 82 is opened.

When the second switch 83 is pressed, power is supplied to the first high-frequency electric path which supplies high-frequency current to the probe distal end 11a of the ultrasonic probe 11, and to the second high-frequency electric path which supplies high-frequency current to the jaw body 28 of the sheath unit 5. Thereby, two bipolar electrodes for high-frequency therapeutic treatment are constituted by the probe distal end 11a of the ultrasonic probe 11 and the jaw body 28 of the sheath unit 5. By supplying high-frequency current between the two bipolar electrodes, i.e. the probe distal end 11a of the ultrasonic probe 11 and the jaw 17 of the sheath unit 5, a high-frequency therapeutic treatment by the bipolar electrodes can be conducted on the living body tissue between the jaw 17 and the probe distal end 11a of the ultrasonic probe 11.

As regards the second switch 83, the same switching operation as with the first switch 82 is performed. Thereby, the metal dome 94 of the second switch 83 is resiliently deformed in a direction in which the metal dome 94 is collapsed. Thus, the second switch contact 91 of the second switch 82 and the second common contact 93 are connected, and the switch contact 91 of the second switch 83 is closed. Accordingly, a signal for driving equipment is generated from the second switch 83 of the switch board 86.

In the case where the movable handle 22 is opened relative to the stationary handle 20, the driving pipe 19 is pulled to the proximal side in interlock with the opening operation of the movable handle 22. In interlock with the pulling operation of the driving pipe 19, the jaw 17 is driven in a direction away from the probe distal end 11a of the ultrasonic probe 11 (“open position”).

The following advantageous effects can be obtained by the above-described structure. Specifically, the handpiece 1 of the present embodiment is provided with the support portion 88 which supports the pressure reception portion 97 of the first switch 82 such that the pressure reception portion 97 is pivotable about the pivotal center O within the operation unit housing 61. Thereby, in the case where a part of the arcuate push button of the pressure reception portion 97 of the first switch 82, which deviates from the central part of the arcuate push button, is pressed, the pressure reception portion 97 can be rotated about the pivotal center O within the operation unit housing 61. By rotating the pressure reception portion 97 relative to the operation unit housing 61, the pressure reception portion 97 can be moved to a position where the surgeon can easily push the first switch contact 90 of the first switch 82. Therefore, the pressure reception portion 97 can be pushed with the attitude of the finger, which enables easy pressing. As a result, the operability and the ease in pressing in performing the switch operation of the first switch 82 can be improved, compared to the case of the conventional medial equipment, in which the operator, at the time of pressing the switch, moves the finger to the position where the switch is disposed and then presses the operation rod of the switch straight in the axial direction with the form of the finger as such.

Furthermore, in the present embodiment, the pressure reception portion 97 of each of the first switch 82 and second switch 83 is formed of the arcuate push button with the shape of the press surface which is elongated in the two directions, i.e. the first direction and the second direction. No matter which position of the arcuate push button of the pressure reception portion 97 of the first switch 82 or second switch 83 is pressed, it is possible to perform the associated switch operation, that is, the operation of generating the signal for driving equipment from the first switch 82 or second switch 83 of the switch board 86. Thus, at the time of the operation of the first switch 82 or second switch 83, even if the user executes a pushing operation in a direction away from the central position of the pressure reception portion 97, the switch operation can be performed. Therefore, the operability of the user can be improved, and the stable switch operation can be performed.

Moreover, in the present embodiment, at the time of pushing the pressure reception portion 97 of the first switch 82, when the metal dome 94 of the first switch 82 is resiliently deformed in the direction in which the metal dome 94 is collapsed, click sound is produced and click sensation is created. Therefore, the switch operation of the first switch 82 can exactly be confirmed.

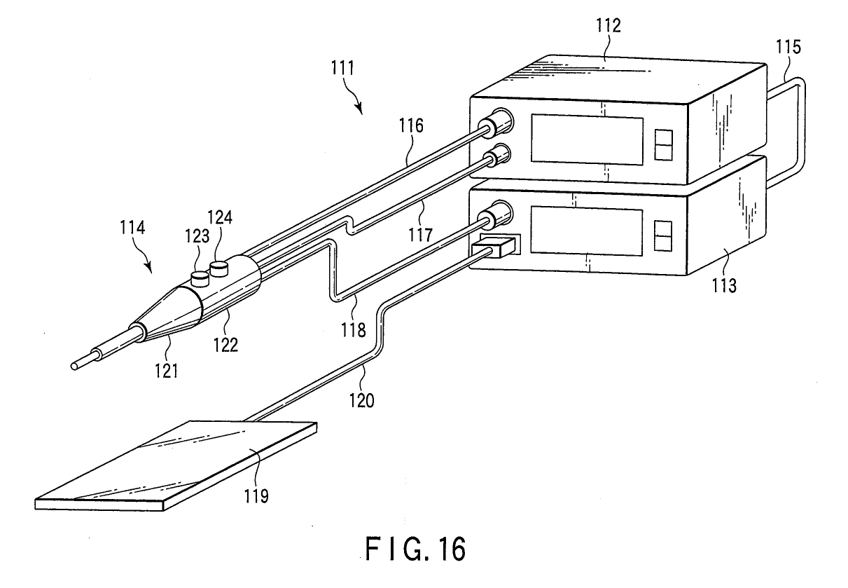

FIG. 16 to FIG. 18 show a second embodiment of the present invention. In the present embodiment, there is provided surgical equipment 111 having a structure which is different from the structure of the surgical operation apparatus according to the first embodiment (see FIG. 1 to FIG. 15).

The surgical equipment 111 mainly comprises an ultrasonic driving apparatus 112, a high-frequency driving apparatus 113, and a handpiece 114. The ultrasonic driving apparatus 112 and the high-frequency driving apparatus 113 are connected over a communication cable 115.

The handpiece 114 is connector-coupled to the ultrasonic driving apparatus 112 over an output connection cable 116 and a SW connection cable 117. Further, the handpiece 114 is connector-coupled to the high-frequency driving apparatus 113 over an output connection cable 118. A counter-electrode plate 119 is connector-coupled to the high-frequency driving apparatus 113 over a connection cable 120.

The handpiece 114 includes a sheath body 121 which is disposed on the distal end side, and a substantially circular-cylindrical operation unit housing 122 which is disposed on the rear end side. The operation unit housing 122 and sheath body 121 are coupled by engagement. The operation unit housing 122 includes two housing parts (left housing part 122a and right housing part 122b) which can be divided to the left and right parts. The left housing part 122a and right housing part 122b are detachably coupled.

In the operation unit housing 122, an ultrasonic transducer (not shown) which generates ultrasonic vibration (e.g. bolt-clamped Langevin type transducer) is fixed. Power for ultrasonic driving is supplied to the ultrasonic transducer through the output connection cable 116.

A probe 125 for transmitting ultrasonic vibration is fastened by a screw to the distal end side of the ultrasonic transducer. A therapeutic treatment section 126 for performing therapeutic treatment of a living body tissue is provided on the distal end side of the probe 125.

In addition, the operation unit housing 122 is provided with a first switch 123 and a second switch 124 functioning as hand switches. The first switch 123 and second switch 124 are mounted on a switch board 131 which is an electric circuit board, and are accommodated in the operation unit housing 122. A conductor wire in the SW connection cable 117 is connected by, e.g. soldering, to a connection terminal of the switch board 131. The SW connection cable 117 extends out of the rear end side of the operation unit housing 122, and is connector-coupled to the ultrasonic driving apparatus 112. Thereby, electric signals of the first switch 123 and second switch 124 are sent to the ultrasonic driving apparatus 112 over the SW connection cable 117.

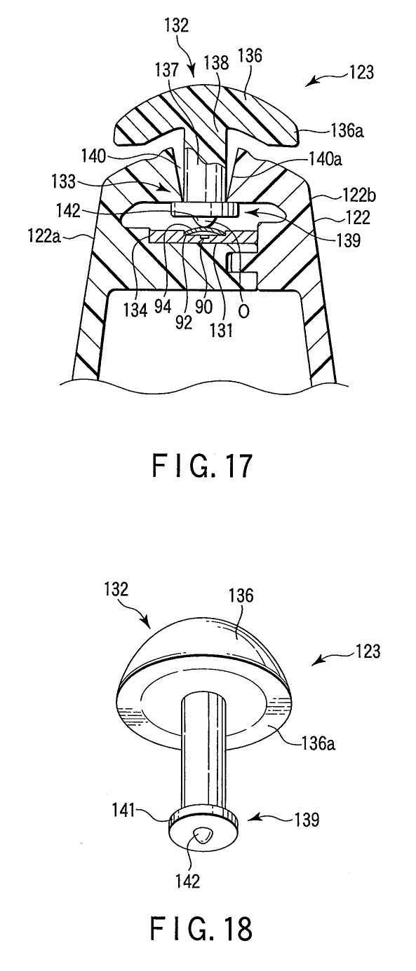

FIG. 17 schematically shows the structure of the first switch 123 of the handpiece 114. The second switch 124 has the same structure as the first switch 123. Thus, only the structure of the first switch 123 is described here, and a description of the structure of the second switch 124 is omitted.

The first switch 124 includes a switch board (signal generation section) 131, a switch operation section 132 and a support portion 133. The switch board 131 has the same structure as the switch board 86 of the first embodiment. Thus, the parts common to those of the switch board 86 of the first embodiment are denoted by like reference numerals, and a description thereof is omitted.

The operation unit housing 122 is provided with a board hold portion 134 which holds the switch board 131, and a switch operation section hold portion 135 which holds the switch operation section 132. The board hold portion 134 is disposed in the state in which the switch board 131 is held at the position corresponding to the first switch 123 and second switch 124 in the operation unit housing 122.

The switch operation section 132 includes a pressure reception portion 136 which is pressed by the user. The support portion 133 supports the pressure reception portion 136 such that the pressure reception portion 136 is pivotable about a pivotal center O within the operation unit housing 122, relative to the operation unit housing 122. Further, the support portion 133 supports the pressure reception portion 136 such that the pressure reception portion 136 can be pushed against the metal dome 94 in the direction of the pivotal center O of the pressure reception portion 136, when the user presses the pressure reception portion 136. When the pressure reception portion 136 is not pressed, the support portion 133 holds the switch operation section 132 at such a position that the switch contact 90 is kept in the open state.

The switch operation section 132 includes a shaft portion 137. A first coupling portion 138, which is coupled to the pressure reception portion 136, is disposed on one end side of the shaft portion 137. A second coupling portion 139, which is coupled to the operation unit housing 122 on the pivotal center O side, is disposed on the other end side of the shaft portion 137.

The support portion 133 includes a gap forming portion 140 which forms, on the first coupling portion 138 side of the switch operation section 132, a gap for making the pressure reception portion 136 movable in the pivotal direction about the pivotal center O of the pressure reception portion 136. The gap forming portion 140 includes a conical inclined surface 140a with an opening gradually increasing on the pressure reception portion 136 side, compared to the pivotal center O side.

FIG. 17 shows the state in which the pressure reception portion 136 of the first switch 123 is held in a non-pressed state, and the shaft portion 137 of the pressure reception portion 136 is held in a neutral position. The shaft portion 137 of the pressure reception portion 136 is held in the state in which the shaft portion 137 can be inclined in an arbitrary direction from the neutral position.

The pressure reception portion 136 of the first switch 123 is a push button with a substantially dome-shaped press surface. The first coupling portion 138 with the shaft portion 137 is disposed at a central position of the dome shape of the pressure reception portion 136. The pressure reception portion 136 includes a circumferential extension portion 136a which extends in the circumferential direction from the first coupling portion 138.

The second coupling portion 139 includes a flange-shaped projection 141 which is projectingly provided on the outer peripheral surface of the shaft portion 137, and a switch contact portion 142 which is in contact with the metal dome 94 of the switch board 131.

In the case where the pressure reception portion 136 of the first switch 123 is operated and pushed straight at the neutral position in FIG. 17, the switch contact portion 142 presses the metal dome 94 of the switch board 131. Thereby, the metal dome 94 of the first switch 123 is resiliently deformed in a direction in which the metal dome 94 is collapsed. Thus, the first switch contact 90 of the first switch 123 and the first common contact 92 are connected, and the switch contact 90 of the first switch 123 is closed. Accordingly, a signal for driving equipment is generated from the first switch 123 of the switch board 131.

In the case where an end edge portion in an arbitrary direction of the circumferential extension portion 136a of the pressure reception portion 136 is pressed, the pressure reception portion 136 of the first switch 123 is inclined in the pressing direction. In this case, by the inclining of the shaft portion 137, the projection 141 abuts upon the inner surface of the operation unit housing 122 at an end edge portion on the side opposite to the pressing direction, thus creating a pivot point. At this time, in accordance with the pivotal movement of the shaft portion 137 about the pivotal point, the switch contact portion 142 presses the metal dome 94 of the switch board 131. Thereby, the metal dome 94 of the first switch 123 is resiliently deformed in a direction in which the metal dome 94 is collapsed. Thus, the first switch contact 90 of the first switch 123 and the first common contact 92 are connected, and the switch contact 90 of the first switch 123 is closed. Accordingly, a signal for driving equipment is generated from the first switch 123 of the switch board 131.

As regards the second switch 124, the same switching operation as with the first switch 123 is performed. Thereby, the metal dome 94 of the second switch 124 is resiliently deformed in a direction in which the metal dome 94 is collapsed. Thus, the second switch contact 91 of the second switch 124 and the second common contact 93 are connected, and the switch contact 91 of the second switch 124 is closed. Accordingly, a signal for driving equipment is generated from the second switch 124 of the switch board 131.

The following advantageous effects can be obtained by the above-described structure. Specifically, the handpiece 114 of the surgical equipment 111 of the present embodiment is provided with the support portion 133 which supports the pressure reception portion 97 of the first switch 123 such that the pressure reception portion 136 is pivotable about the pivotal center O within the operation unit housing 122. Thereby, in the case where a part of the dome-shaped push button of the pressure reception portion 136 of the first switch 123, which deviates from the central part of the push button, is pressed, the pressure reception portion 136 can be rotated about the pivotal center O within the operation unit housing 122. By rotating the pressure reception portion 136 relative to the operation unit housing 122, the pressure reception portion 136 can be moved to a position where the surgeon can easily push the first switch contact 90 of the first switch 123. Therefore, the pressure reception portion 136 can be pushed with the attitude of the finger, which enables easy pressing. As a result, the operability and the ease in pressing in performing the switch operation of the first switch 123 can be improved.

Furthermore, in the present embodiment, the pressure reception portion 136 of each of the first switch 123 and second switch 124 is formed of the push button with the dome-shaped press surface. No matter which position of the dome-shaped push button of the pressure reception portion 136 of the first switch 123 or second switch 124 is pressed, it is possible to perform the associated switch operation, that is, the operation of generating the signal for driving equipment from the first switch 123 or second switch 124 of the switch board 131. Thus, at the time of the operation of the first switch 123 or second switch 124, even if the user executes a pushing operation in a direction away from the central position of the pressure reception portion 136, the switch operation can be performed. Therefore, the operability of the user can be improved, and the stable switch operation can be performed.

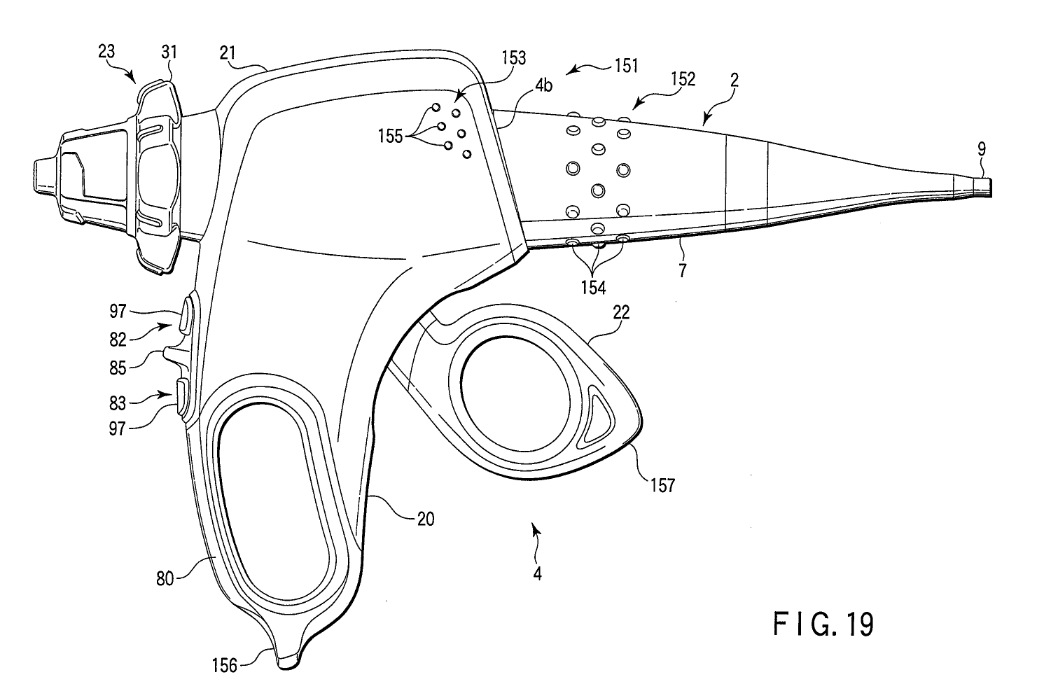

FIG. 19 shows a handpiece 151 of a surgical operation apparatus according to a third embodiment of the present invention. The handpiece 151 of the surgical operation apparatus of the present embodiment is an ultrasonic coagulation/incision therapeutic apparatus which is similar to the apparatus of the first embodiment (see FIG. 1 to FIG. 15). The main structure is the same as that of the first embodiment, and specific structural parts are different from the first embodiment. In FIG. 19, the parts common to those in the first embodiment are denoted by like reference numerals, and a description thereof is omitted.

Specifically, in the present embodiment, external display sections 152 and 153 having similar kinds of design are provided on assembly parts between the transducer unit 2 and the handle unit 4. The first external display section 152 of the transducer unit 2 is configured such that a plurality of dot-shaped projections 154 are juxtaposed along the circumferential direction on the outer peripheral surface of a distal end portion of the transducer cover 7.

The second external display section 153 of the handle unit 4 is disposed near a transducer connection part 4b of the hold cylinder 21. The second external display section 153 is configured such that a plurality of dot-shaped projections 155 are juxtaposed on both side surfaces of a rear end portion of the hold cylinder 21. The first external display section 152 of the transducer unit 2 and the second external display section 153 of the handle unit 4 have common designs so as to have uniformity. Thereby, it can be easily confirmed that these parts are of the same model type.

A claw-shaped finger hook portion 156 is projectingly provided on a lower end portion of the plural-finger insertion ring portion 80 of the stationary handle 20. Similarly, a substantially triangular projection portion 157 is projectingly provided on a terminal end portion of the movable handle 22.

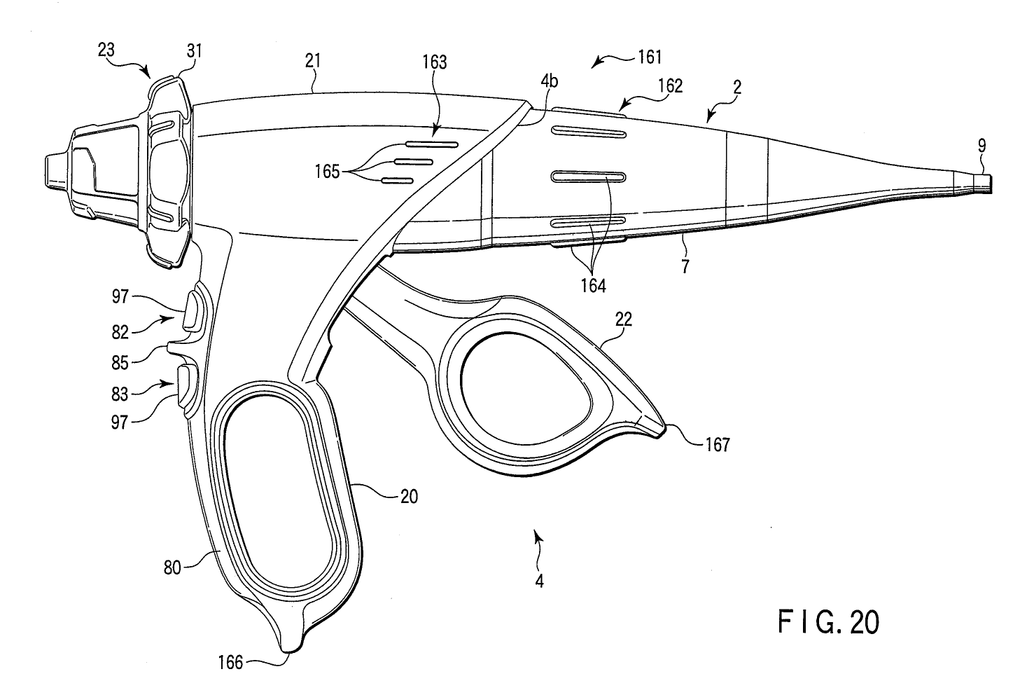

FIG. 20 shows a handpiece 161 of a surgical operation apparatus according to a fourth embodiment of the present invention. The handpiece 161 of the surgical operation apparatus of the present embodiment is an ultrasonic coagulation/incision therapeutic apparatus which is similar to the apparatus of the first embodiment (see FIG. 1 to FIG. 15). The main structure is the same as that of the first embodiment, and specific structural parts are different from the first embodiment. In FIG. 20, the parts common to those in the first embodiment are denoted by like reference numerals, and a description thereof is omitted.

Specifically, in the present embodiment, external display sections 162 and 163 having similar kinds of design are provided on assembly parts between the transducer unit 2 and the handle unit 4. The shapes of these external display sections 162 and 163 are different from the shapes of the external display sections 152 and 153 of the handpiece 151 of the third embodiment (see FIG. 19).

The first external display section 162 of the transducer unit 2 is configured such that a plurality of linear projections 164 are juxtaposed along the circumferential direction on the outer peripheral surface of a distal end portion of the transducer cover 7. The respective projections 164 extend substantially parallel to the axial direction.

The second external display section 163 of the handle unit 4 is disposed near a transducer connection part 4b of the hold cylinder 21. The second external display section 163 is configured such that a plurality of linear projections 165 are juxtaposed on both side surfaces of a rear end portion of the hold cylinder 21. The first external display section 162 of the transducer unit 2 and the second external display section 163 of the handle unit 4 have common designs so as to have uniformity. Thereby, it can be easily confirmed that these parts are of the same model type.

A claw-shaped finger hook portion 166 is projectingly provided on a lower end portion of the plural-finger insertion ring portion 80 of the stationary handle 20. Similarly, a claw-shaped finger hook portion 167 is projectingly provided on a terminal end portion of the movable handle 22.

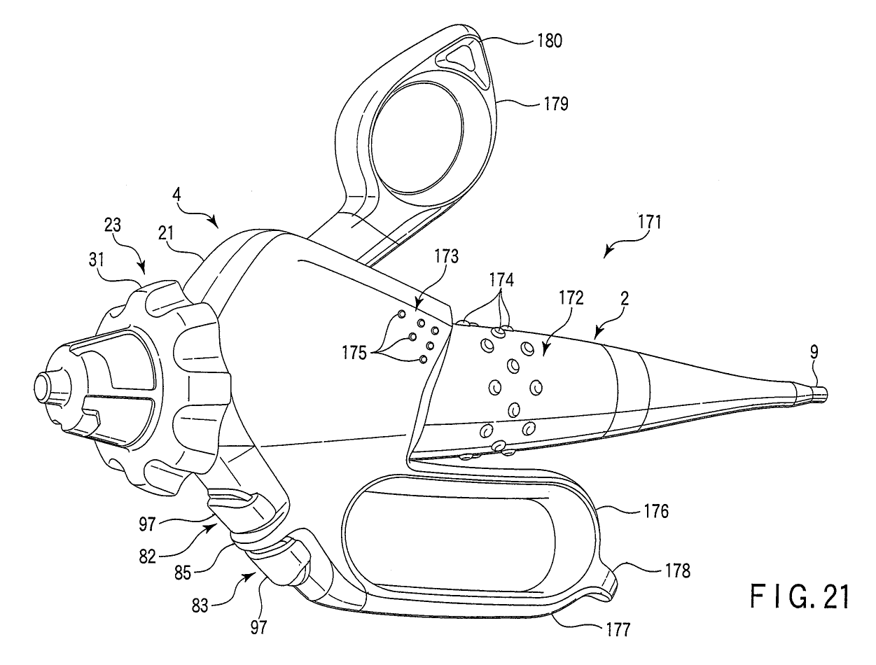

FIG. 21 shows a handpiece 171 of a surgical operation apparatus according to a fifth embodiment of the present invention. The present embodiment is a modification of the handpiece 151 of the third embodiment (see FIG. 19). The handpiece 171 of the surgical operation apparatus according to the present embodiment is an ultrasonic coagulation/incision therapeutic apparatus which is similar to the apparatus of the first embodiment (see FIG. 1 to FIG. 15). The main structure is the same as that of the first embodiment, and specific structural parts are different from the first embodiment. In FIG. 21, the parts common to those in the first embodiment are denoted by like reference numerals, and a description thereof is omitted.

Specifically, in the present embodiment, external display sections 172 and 173 having similar kinds of design are provided on assembly parts between the transducer unit 2 and the handle unit 4. The first external display section 172 of the transducer unit 2 has substantially the same structure as the first external display section 152 of the transducer unit 2 of the handpiece 151 according to the third embodiment (see FIG. 19). The first external display section 172 of the transducer unit 2 is configured such that a plurality of dot-shaped projections 174 are juxtaposed along the circumferential direction on the outer peripheral surface of a distal end portion of the transducer cover 7.

The second external display section 173 of the handle unit 4 is disposed near a transducer connection part 4b of the hold cylinder 21. The second external display section 173 is configured such that a plurality of dot-shaped projections 175 are juxtaposed on both side surfaces of a rear end portion of the hold cylinder 21. The first external display section 172 of the transducer unit 2 and the second external display section 173 of the handle unit 4 have common designs so as to have uniformity. Thereby, it can be easily confirmed that these parts are of the same model type. The second external display section 173 of the handle unit 4 of the handpiece 171 according to the present embodiment and the second external display section 153 of the handle unit 4 of the handpiece 151 according to the third embodiment are formed with different patterns.

In the present embodiment, the structure of the handle unit 4 differs from the structure of the handle unit 4 of the third embodiment. Specifically, in the handle unit 4 of the present embodiment, a plural-finger insertion ring portion 177 of a stationary handle 176 is provided to extend rearward under the hold cylinder 21. A claw-shaped finger hook portion 178 is projectingly provided on a terminal end portion of the plural-finger insertion ring portion 80. Similarly, a claw-shaped finger hook portion 167 is projectingly provided on a terminal end portion of the movable handle 22.

A movable handle 179 is provided to extend on the upper side of the hold cylinder 21. A substantially triangular projection portion 180 is projectingly provided on a terminal end portion of the movable handle 179.

The present invention is not limited to the above-described embodiments. For example, the surgical equipment may be a high-frequency surgical apparatus. The high-frequency surgical apparatus includes an electrical connection section which electrically connects a handpiece and a generator which generates driving energy for driving the handpiece, and an electrically conductive probe which is provided in the handpiece and is electrically connected to the generator. Switches, which control a high-frequency electric current that is supplied from the generator to the probe, are composed of the first switch 82 and second switch 83 of the surgical operation apparatus of the first embodiment (see FIG. 1 to FIG. 15). Needless to say, other various modifications may be made without departing from the spirit of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A switching structure which controls driving of equipment, comprising:
a handle unit which is provided on the equipment and is held by a user;
a switch operation section which is provided on the handle unit and includes a pressure reception portion which is pressed by the user; and
a support portion which supports the pressure reception portion such that the pressure reception portion is pivotable about a pivotal center within the handle unit, relative to the handle unit, and also supports the pressure reception portion such that the pressure reception portion can be pushed from a desired direction toward the pivotal center of the pressure reception portion, when the user presses the pressure reception portion.

2. The switching structure according to claim 1, further comprising a signal generation section which includes a switch contact and an urging member which holds the switch contact in an open state, and generates a signal for driving the equipment at a time when the switch contact is operated and closed,
wherein the support portion holds, when the pressure reception portion is not pressed, the switch operation section at such a position that the switch contact is kept in the open state, and
the support portion supports, when the pressure reception portion is pressed, the switch operation section such that the switch operation section can be moved against the urging member in such a direction as to close the switch contact.

3. The switching structure according to claim 1, wherein the switch operation section includes a shaft portion, a first coupling portion which is disposed on one end side of the shaft portion and is coupled to the pressure reception portion, and a second coupling portion which is disposed on the other end side of the shaft portion an is coupled to the handle unit on the pivotal center side, and
the support portion includes a gap forming portion which forms, on the first coupling portion side, a gap for making the pressure reception portion movable in a pivotal direction about the pivotal center of the pressure reception portion.

4. The switching structure according to claim 3, wherein the gap forming portion includes an inclined surface with an opening gradually increasing on the pressure reception portion side, compared to the pivotal center side.

5. The switching structure according to claim 1, wherein the switch operation section pivots in a direction of pressing by the finger of the user who presses the pressure reception portion.

6. The switching structure according to claim 1, wherein the switch operation section is pivotable in a range of movement of the finger of the user who presses the pressure reception portion.

7. The switching structure according to claim 3, wherein the switch operation section is configured such that the pressure reception portion is pivotable in a state in which the shaft portion is inclined at least in two directions, i.e. a first direction and a second direction, from a neutral position of the shaft portion,
the second coupling portion includes a first projection which projects in the first direction, a second projection which projects in the second direction, and a switch contact portion which is in contact with the signal generation section,
the first projection abuts upon an inner surface of the handle unit in a case where the pressure reception portion is pivoted in the second direction, thus creating a first pivot point, and the switch contact portion presses the signal generation section in such a direction as to close the switch contact, in accordance with pivotal movement of the shaft portion about the first pivotal point, and
the second projection abuts upon the inner surface of the handle unit in a case where the pressure reception portion is pivoted in the first direction, thus creating a second pivot point, and the switch contact portion presses the signal generation section in such a direction as to close the switch contact, in accordance with pivotal movement of the shaft portion about the second pivotal point.

8. The switching structure according to claim 7, wherein a press surface of the pressure reception portion has an arcuate shape along two directions, i.e. the first direction and the second direction.

9. The switching structure according to claim 1, wherein there are provided a plurality of said switch operation sections, and
the handle unit is provided with a finger rest portion on which the user places the finger between the pressure reception portions of the plurality of switch operation sections.

10. The switching structure according to claim 3, wherein the switch operation section is configured such that the pressure reception portion is pivotable in a state in which the shaft portion is inclined in an arbitrary direction from a neutral position of the shaft portion,
the second coupling portion includes a projection which projects in a circumferential direction of the shaft portion, and a switch contact portion which is in contact with the signal generation section,
an end portion of the projection, which is located on a side opposite to a pivotal direction of the pressure reception portion, abuts upon an inner surface of the handle unit, thus creating a pivotal point, in a case where the pressure reception portion is pivoted in a direction other than a direction of a neutral position of the shaft portion, and the switch contact portion presses the signal generation section in such a direction as to close the switch contact, in accordance with pivotal movement of the shaft portion about the pivotal point.

11. Surgical equipment including a handpiece having a handle unit which is held by a surgeon, and a switch which is provided on the handle unit and controls driving of the equipment, wherein the switch includes:
a switch operation section which includes a pressure reception portion which is pressed by a user; and
a support portion which supports the pressure reception portion such that the pressure reception portion is pivotable about a pivotal center within the handle unit, relative to the handle unit, and also supports the pressure reception portion such that the pressure reception portion can be pushed from a desired direction toward the pivotal center of the pressure reception portion, when the user presses the pressure reception portion.

12. The surgical equipment according to claim 11, further comprising a signal generation section which includes a switch contact and an urging member which holds the switch contact in an open state, and generates a signal for driving the equipment at a time when the switch contact is operated and closed,
wherein the support portion holds, when the pressure reception portion is not pressed, the switch operation section at such a position that the switch contact is kept in the open state, and
the support portion supports, when the pressure reception portion is pressed, the switch operation section such that the switch operation section can be moved against the urging member in such a direction as to close the switch contact.

13. The surgical equipment according to claim 12, wherein the surgical equipment is an ultrasonic surgical apparatus comprising:
an electrical connection section which electrically connects the handpiece and a generator which generates driving energy for driving the handpiece;
an ultrasonic transducer which is provided in the handpiece and is electrically connected to the generator; and
an ultrasonic probe which is connected to the ultrasonic transducer and to which ultrasonic vibration from the ultrasonic transducer is transmitted.

14. The surgical equipment according to claim 13, further comprising:
a sheath which covers the ultrasonic probe; and
a jaw which is attached to a distal end portion of the sheath such that the jaw is pivotable relative to the distal end portion of the ultrasonic probe.

15. The surgical equipment according to claim 11, wherein the surgical equipment is a high-frequency surgical apparatus comprising:
an electrical connection section which electrically connects the handpiece and a generator which generates driving energy for driving the handpiece; and
an electrically conductive probe which is provided in the handpiece and is electrically connected to the generator,
wherein a high-frequency current, which is supplied from the generator to the probe, is controllable by the switch.

16. The surgical equipment according to claim 11, wherein the surgical equipment is a surgical operation apparatus comprising:
an electrical connection section which electrically connects the handpiece and a generator which generates driving energy for driving the handpiece;
an ultrasonic transducer which is provided in the handpiece and is electrically connected to the generator; and
an electrically conductive probe which is connected to the ultrasonic transducer and is electrically connected to the generator,
wherein a high-frequency current, which is supplied from the generator to the probe, and a driving current for ultrasonic, which is supplied to the ultrasonic transducer, are controllable by the switch.
